(19) United States
(12) Patent Application Publication
(10) Pub. No.: US 2003/0191081 A1
(43) Pub. Date: Oct. 9, 2003

(54) Pharmaceutical compositions and methods of use thereof comprising polyanionic polymers and amphiphilic block copolymers to improve gene expression

(76) Inventors: Pierre Lemieux, Ste-Therese (CA); Phillipe Goyette, Montreal (CA)

Correspondence Address: Mathews, Collins, Shepherd & McKay, P.A., 100 Thanet Circle, Suite 306, Princeton, NJ, 08540, US

(21) Appl. No.: 10/329,646

(22) Filed: Dec. 26, 2002

Related U.S. Application Data
(60) Provisional application No. 60/344,075, filed on Dec. 28, 2001.

(51) Int. Cl. A61K 48/00; A61K 9/14

(52) U.S. Cl. 514/44; 424/486

(57) ABSTRACT

The present invention is directed to polynucleotide compositions containing (a) a polynucleotide or derivative thereof and (b) a block copolymer having a polyether segment and a polyanion segment, compositions containing (a) a polynucleotide or its derivative thereof, (b) at least one polyanionic polymer, and (c) at least one amphiphilic block copolymer, and methods of using these compositions for gene therapy.

This application claims the benefit of U.S. Provisional Application No. 60/344,075, filed on Dec. 28, 2001, the content of which is hereby incorporated by reference.

### FIELD OF THE INVENTION

The invention relates to the field of gene delivery, such as gene therapy and genetic vaccination.

### BACKGROUND OF THE INVENTION

The unique features of smooth, skeletal, and cardiac muscles, have presented numerous challenges for the development and administration of effective polynucleotide compositions for intramuscular administration. Direct injection of purified plasmids (“naked DNA”) in isotonic saline into muscle was found to result in DNA uptake and gene expression in smooth, skeletal, and cardiac muscles of various species. Rolland A., Critical Reviews in Therapeutic Drug Carrier Systems, Begell House, 143 (1998). It is believed that the unique cytoarchitectural features of muscle tissue are responsible for the uptake of polynucleotides because skeletal and cardiac muscle cells appear to be better suited to take-up and express injected foreign DNA vectors relative to other types of tissues. Dowty & Wolff, Gene Therapeutics: Methods and Applications of Direct Gene Transfer, Birkhäuser, Boston, p. 182 (1994). The relatively low expression levels attained by this method, however, have limited its applications. See Aihara and Miyazaki, Nature Biotechnology, 16:867 (1998). Additionally, traditional gene delivery systems such as polycations, cationic liposomes, and lipids that are commonly proposed to boost gene expression in other tissues usually result in inhibition of gene expression in skeletal and cardiac muscles. Dowty & Wolff, Gene Therapeutics: Methods and Applications of Direct Gene Transfer, Birkhäuser, Boston, p. 82 (1994).

Anionic polymers such as dextran sulfate and salmon DNA can decrease gene expression in the muscle. Rolland A., Critical Reviews in Therapeutic Drug Carrier Systems, Begell House, 1998, p. 143. Various noncondensive interactive polymers have been used with a limited success to modify gene expression in striated muscle. Nonionic polymers such as poly(vinyl pyrrolidone) poly(vinyl alcohol) interact with plasmids through hydrogen bonding. Rolland A., Critical Reviews in Therapeutic Drug Carrier Systems, Begell House, 1998, p. 143. These polymers may facilitate the uptake of polynucleotides in muscle cells and cause up to 10-fold enhancement of gene expression. However, to achieve a significant increase in gene expression, high concentrations of polymers (about 5% and more) need to be administered. Mumper et al., Pharmacol. Res., 13, 701-709 (1996); March et al., Human Gene Therapy, 6(1), 41-53 (1995). This high concentration of poly(vinyl pyrrolidone) poly(vinyl alcohol) needed to improve gene expression can be associated with toxicity, inflammation, and other adverse effects in muscle tissues. Block copolymers have been used to improve gene expression in muscle or to modify the physiology of the muscle for subsequent therapeutic applications. See U.S. Pat. Nos. 5,552,309; 5,470,568; 5,605,687; and 5,824,322. For example, block copolymers can be used in a gel-like form (more than 1% of block copolymers) to formulate virus particles used to perform gene transfer in the vasculature. In the same range of block copolymers concentration (1-10%), it is possible with block copolymer to modify the permeability of damaged muscle tissue (radiation and electrical injury, and frost bite). In addition DNA molecules can be incorporated into cells following membrane permeabilization with block copolymers. For these applications, block copolymers were used at concentrations giving gel-like structures and viscous delivery systems. These systems are unlikely to enable diffusion of the DNA injected into the muscle, however, thus limiting infusion of the DNA into the myofibers.

There is thus a need for compositions and methods increasing efficacy of polynucleotides expression upon administration in the muscle.

Beside the need to improve gene expression in muscle other tissues in the body would benefit from a gene transfer in a situation when there is a genetic disorder, and/or an abnormal over-expression of a gene, and/or absence of a normal gene. Several polynucleotides such as RNA, DNA, viruses, ribozymes can be used for gene therapy purposes. However, many problems, like the ones described below, have been encountered for successful gene therapies.

Conventional methods have included use of polyglutamate plus electroporation (polyanionic) to help gene expression in muscle. The disadvantage of polyglutamate is that it does not improve the poloxamers effect on DNA expression and electroporation produces serious tissue damage.

The use of antisense polynucleotides to treat genetic diseases, cell mutations (including cancer causing or enhancing mutations) and viral infections has gained widespread attention. This treatment tool is believed to operate, in one aspect, by binding to “sense” strands of mRNA encoding a protein believed to be involved in causing the disease site sought to be treated, thereby stopping or inhibiting the translation of the mRNA into the unwanted protein. In another aspect, genomic DNA is targeted for binding by the antisense polynucleotide (forming a triple helix), for instance, to inhibit transcription. See Helene, Anti-Cancer Drug Design, 6:569 (1991). Once the sequence of the mRNA sought to be bound is known, an antisense molecule can be designed that binds the sense strand by the Watson-Crick base-pairing rules, forming a duplex structure analogous to the DNA double helix. Gene Regulation: Biology of Antisense RNA and DNA, Erikson and lxzant, eds., Raven Press, New York, 1991; Helene, Anti-Cancer Drug Design, 6:569 (1991); Crooke, Anti-Cancer Drug Design, 6:609 (1991). A serious barrier to fully exploiting this technology is the problem of efficiently introducing into cells a sufficient number of antisense molecules to effectively interfere with the translation of the targeted mRNA or the function of DNA.

### SUMMARY OF THE INVENTION

The invention relates to compositions and methods of use thereof comprising polyanionic polymers and amphiphilic block copolymers. These compositions are useful for gene therapy purposes, including gene replacement or excision therapy, and gene addition therapy, vaccination, as well as therapeutic situations in which it is desirable to express or down-regulate a polypeptide in the body or in vitro.

The contents of the patents and publications cited herein and the contents of documents cited in these patents and publications are hereby incorporated herein by reference to the extent permitted.

### BRIEF DESCRIPTION OF THE FIGURES

FIG. 1A is a graph of luciferase activity for CpG ODN phosphodiester.

FIG. 1B is a graph of luciferase activity for non-CpG ODN phosphorothio.

FIG. 2 is a graph of poloxamers with polyacrylic acid.

### DETAILED DESCRIPTION OF THE INVENTION

Definitions

As used herein, the terms below have the following meaning:

Backbone: Used in graft copolymer nomenclature to describe the chain onto which the graft is formed.

Block copolymer: A combination of two or more chains of constitutionally or configurationally different features.

Branched polymer: A combination of two or more chains linked to each other, in which the end of at least one chain is bonded at some point along the other chain.

Chain: A polymer molecule formed by covalent linking of monomeric units.

Configuration: Organization of atoms along the polymer chain, which can be interconverted only by the breakage and reformation of primary chemical bonds.

Conformation: Arrangements of atoms and substituents of the polymer chain brought about by rotations about single bonds.

Copolymer: A polymer that is derived from more than one species of monomer.

Cross-link: A structure bonding two or more polymer chains together.

Dendrimer: A regularly branched polymer in which branches start from one or more centers.

Dispersion: Particulate matter distributed throughout a continuous medium.

Graft copolymer: A combination of two or more chains of con-stitutionally or configurationally different features, one of which serves as a backbone main chain, and at least one of which is bonded at some points along the backbone and constitutes a side chain.

Homopolymer: Polymer that is derived from one species of monomer.

Link: A covalent chemical bond between two atoms, including bond between two monomeric units, or between two polymer chains.

Polymer blend: An intimate combination of two or more polymer chains of constitutionally or configurationally different features, which are not bonded to each other.

Polymer fragment (or Polymer segment): A portion of polymer molecule in which the monomeric units have at least one constitutional or configurational feature absent from adjacent portions.

Polynucleotide: A natural or synthetic nucleic acid. Generally polynucleotides can be oligonucleotide, DNA, RNA, cDNA, a DNA fragment cloned in a DNA vector, a DNA fragment cloned in DNA vector and viral vector and plasmid vector. Also included among the suitable polynucleotides are viral genomes and viruses (including the lipid or protein viral coat). Viral vectors include, but not limited to, retroviruses, adenoviruses, herpes-virus, or Pox-virus. Other suitable viral vectors for use with the present invention will be obvious to those skilled in the art. Preferably, the polynucleotide has at least about 3 bases, more preferably at least about 5 bases and the most preferably at least 10 bases.

The polynucleotide may include a promoter, enhancer and other cis-acting control regions that provide a desired level and specificity of expression in the cells of a region operably linked thereto that encodes an RNA, such as an anti-sense RNA, or a protein. The polynucleotides may also contain several such operably linked control and encoding regions for expression of one or more mRNAs or proteins, or a mixture of the two.

Polynucleotide derivative: A polynucleotide having one or more moieties (i) wherein the moieties are cleaved, inactivated or otherwise transformed so that the resulting material can function as a polynucleotide, or (ii) wherein the moiety does not prevent the derivative from functioning as a polynucleotide.

Repeating unit: Monomeric unit linked into a polymer chain.

Side chain: The grafted chain in a graft copolymer.

Starblock copolymer: Three or more chains of different constitutional or configurational features linked together at one end through a central moiety.

Star polymer: Three or more chains linked together at one end through a central moiety.

Surfactant: Surface active agent that is adsorbed at interface.

Viral vector: A construct derived from a virus and used in gene transfer.

Preferred embodiments include compositions having polynucleotides and block copolymers with anionic segments. In one embodiment, for intramuscular administration, polynucleotides are formulated with block copolymers of poly(oxyethylene) and poly(oxypropylene). In another embodiment, the composition contains (a) a polynucleotide or its derivative thereof, (b) at least one polyanionic polymer, and (c) at least one amphiphilic block copolymer. Preferably the at least one amphiphilic block copolymer is a block copolymer of poly(oxyethylene) and poly(oxypropylene) and more preferably the at least one amphiphilic block copolymer contains at least one poly(oxyethylene) and poly(oxypropylene) block copolymer with oxyethylene content of 50% or less, and at least one poly(oxyethylene) and poly(oxypropylene) block copolymer with oxyethylene content of 50% or more.

The compositions of the current invention provide an efficient vehicle for introducing polynucleotides into a cell, protecting polynucleotides against degradation in body fluids, transport of polynucleotides across biological membranes and biological barriers (such as the blood-brain barrier, blood-cerebral fluid barrier, and intestinal barrier), modification of biodistribution of polynucleotides in the body and enhancement of gene expression including selective gene expression in various tissues and organs in the body of human or animal.

In a preferred embodiment, the present invention provides a method of delivering a polynucleotide to a cell comprising administering a composition containing (a) a polynucleotide or its derivative thereof, (b) at least one polyanionic polymer, and (c) at least one amphiphilic block copolymer. The present invention also provides a method of delivering a polynucleotide to a cell comprising administering a composition containing (a) a polynucleotide or derivative thereof and (b) a block copolymer having a polyether segment and a polyanion segment.

The compositions of the invention can be administered orally, topically, rectally, vaginally, parenterally, intramuscularly, intradermally, subcutaneously, intraparitoneally, or intravenously, or by pulmonary route by use of an aerosol, or parenterally, i.e. intramuscularly, subcutaneously, intraperitonealily or intravenously. The compositions can be administered alone, or it can be combined with a pharmaceutically-acceptable carrier or excipient according to standard pharmaceutical practice. For the oral mode of administration, the compositions can be used in the form of tablets, capsules, lozenges, troches, powders, syrups, elixirs, aqueous solutions and suspensions, and the like. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the compositions can be combined with emulsifying and suspending agents. If desired, certain sweetening and/or flavoring agents can be added. For parenteral administration, sterile solutions of the conjugate are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers. For pulmonary administration, diluents and/or carriers are selected to be appropriate to allow the formation of an aerosol.

The invention further relates to methods of delivering polynucleotides into cells utilizing the compositions of the invention, and methods of treatment having administering these compositions in humans and animals.

In another preferred embodiment, the present invention provides a method of treating an animal comprising administering a composition containing (a) a polynucleotide or its derivative thereof, (b) at least one polyanionic polymer, and (c) at least one amphiphilic block copolymer, or a composition containing (a) a polynucleotide or derivative thereof and (b) a block copolymer having a polyether segment and a polyanion segment.

In another preferred embodiment, the present invention provides a method of treating an animal comprising intramuscularly administering a composition containing (a) a polynucleotide or its derivative thereof, (b) at least one polyanionic polymer, and (c) at least one amphiphilic block copolymer, or a composition containing (a) a polynucleotide or derivative thereof and (b) a block copolymer having a polyether segment and a polyanion segment. Preferably, the compositions are administered to at least one of smooth, skeletal, and cardiac muscles.

In another preferred embodiment, the present invention further provides a method of treating an animal comprising intradermally administering a composition containing (a) a polynucleotide or its derivative thereof, (b) at least one polyanionic polymer, and (c) at least one amphiphilic block copolymer, or a composition containing (a) a polynucleotide or derivative thereof and (b) a block copolymer having a polyether segment and a polyanion segment.

In a preferred embodiment, the block copolymer which is combined with the polyanionic polymer conforms to one of the following formulae:

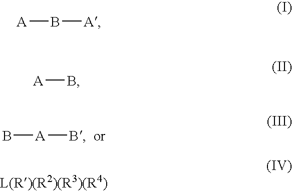

wherein A and A′ are A-type linear polymeric segments, B and B′ are B-type linear polymeric segments, and R¹, R², R³, and R⁴ are either block copolymers of formulas (I), (II), or (III), or hydrogen and L is a linking group, with the proviso that no more than two of R¹, R², R³, or R⁴ are hydrogen.

In another preferred embodiment, the block copolymers are poly(oxyethylene) and poly(oxypropylene) chain segments. In yet another preferred embodiment, the polynucleotide compositions have polyanionic polymers having a plurality of anionic repeating units. In this case, the polynucleotides can be complexed with the polyanion and stabilized in the complex. These compositions demonstrate increased permeability across cell membranes and are well suited for use as vehicles for delivering nucleic acid into cells.

In another embodiment, the invention relates to polynucleotide compositions having:

(a) a polynucleotide or derivative thereof;

(b) a block copolymer having a polyether segment and a polyanion segment, wherein the polyether segment comprises at least an A-type block, and the polyanion segment comprises a plurality of anionic repeating units.

In a preferred second embodiment, the copolymer relates to polymers of formulae:

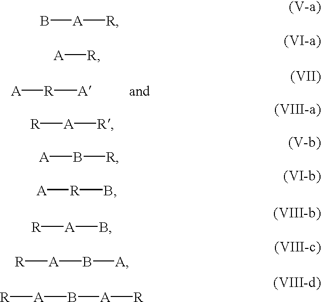

wherein A, A′, and B are as described above, wherein R and R′ are polymeric segments having a plurality of anionic repeating units, and each anionic repeating unit in a segment is the same or different from another unit in the segment. The R and R′, blocks can be termed “R-type” polymeric segments or blocks. The polynucleotide compositions of this embodiment provide an efficient vehicle for introducing polynucleotides into a cell.

Accordingly, the invention thus further relates to methods of inserting polynucleotide into cells utilizing the compositions of the invention.

In yet another embodiment, the invention relates to polynucleotide compositions having a polynucleotide derivative comprising a polynucleotide segment and a polyether segment attached to one or both of the polynucleotide 5′ and 3′ ends, wherein the polyether comprises an A-type polyether segment.

In a preferred third embodiment, the derivative comprises a block copolymer of formulas:

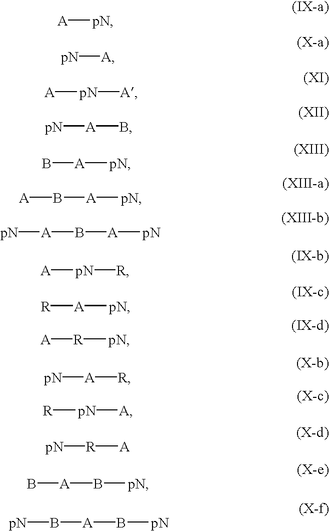

wherein pN represents a polynucleotide having 5′ to 3′ orientation, and A, A′, and B are polyether segments as described above. In another preferred third embodiment, the polynucleotide complex comprises a polyanionic polymer. The polynucleotide component (pN) of formulas (IX) through (XIII) will preferably have from about 5 to about 1,000,000 bases, more preferably about 5 to about 100,000 bases, yet more preferably about 10 to about 10,000 bases.

The polynucleotide compositions provide an efficient vehicle for introducing polynucleotides into a cell. Accordingly, the invention also relates to methods of inserting polynucleotide into cells the compositions of the invention. In another preferred embodiment, polynucleotides are covalently linked to block copolymers of poly(oxyethylene) and poly(oxypropylene).

Preferred polyanion segments, which are combined with poloxamers, comprise at least three of the same or different repeating unites containing at least one atom selected from the group consisting of oxygen, sulfur, or phosphorus. Suitable polyanion fragments are homopolymers or copolymers and the salts thereof which include repeating unites containing carboxylic, sulfonic, sulfuric, phosphoric, or the salts thereof, such carboxylates, sulfonates, sulfates, phosphates, phosphonates and the like have been described in March, “Advanced Organic Chemistry”, 4^{th} edition, 1992, Wiley-Interscience, New York.

Examples of polyanion segments include but are not limited to polymethacrylic acid and its salts; polyacrylic acid and its salts; copolymers of methacrylic acids and its salts; copolymers of acrylic acid and its salts; heparin; poly(phosphate); polyamino acid, such as polyaspartic acid, polylactic acid, and their copolymers, polynucleotides, carboxylated dextran, and the like. Preferred polyanions include the products of polymerization or copolymerization of monomers that polymerize to yield a product having carboyl pendant groups. Representative examples of such monomers are acrylic acid, aspartic acid, 1,4-phenylenediacrylic acid, citracinic acid, citraconic anhydride, trans-cinnamic acid, 4-hydroxy cinnamic acid, trans-glutaconic acid, glutamic acid, itaconic acid, linoleic acid, linolenic acid, methacrylic acid, trans-beta-hydromuconic acid, trans-trans-muconic acid, ricinolei acid, 2-propene-1-sulfonic acid, 4-styrene sulfonic acid, trans-traumatic acid, vinylsulfonic acid, vinyl phosphoric acid, vinyl benzoic acid and vinyl glycolic acid. The polyanion fragments have several ionizable groups that can form net negative charge at physiologic pH. Preferably, the polyanion fragments will have at least about 3 negative charges at physiologic pH, more preferably, at 25 least about 6, still more preferably, at least about 12. Also preferred are polymers or fragments that, at physiologic pH, can present negative charges with about a distances between the charges of about 2 Å to about 10 Å.

Generally, polymer having any degree of polymerization can be used in the present composition as long as it can help to deliver polynucleotide or its derivative to cells. The degree of polymerization of the polymer can range from about 5 to about 10,000,000, preferably from about 10 to about 100,000, more preferably from about 10 to about 10,000, still more preferably from about 10 to about 1000 and the most preferably from about 10 to about 200.

Generally, the polymer can be used in any concentration that can help the delivery of polynucleotide or its derivative to cells. The polymer concentration can ranges from about 0.000001% wt to 20% wt, preferably from about 0.0001% to about 10%, more preferably from about 0.01% to about 1%.

Without intending to be bound by any particular theory of operation, it is believed that the optimal concentration range may depend on the degree of polymerization. It is also believed that the optimal combination of the concentration and degree of polymerization should be chosen to avoid formation of solutions with high viscosity and in particularly, avoid polymer gels, solids and other non-liquid forms. For example, when a polymer having a high degree of polymerization is used, the optimal concentration of the polymer is lower than that of the polymer having a low degree of polymerization as the former increases the viscosity at a faster rate than the latter.

Preferred formulations should form homogeneous or micellar solutions. In one preferred embodiment, formation of large particles of more than one molecule should be avoided, particularly particles with sizes above 300 nm and in some preferred embodiments of particles above 50 nm. The polymer components of the composition including polyanions added and polynucleotide or its derivative may have hydrodynamic diameters exceeding the above size ranges. This does not preclude the use of such formulation. It has also been recognized that poly(ethylene oxide)-poly(propylene oxide) block copolymer components of the formulation can form micelles due to self-assembly. The sizes of these micelles normally are below the above size ranges. The present formulation allows the existence of micelles formed by the block copolymer.

In yet another preferred embodiment, the compositions of the invention are useful for gene therapy purposes, including gene replacement or excision therapy, and gene addition therapy, vaccination, and any therapeutic situation in which a polypeptide should be expressed or down-regulated in the body or in vitro. In one aspect of this invention the polynucleotide compositions are used for gene therapy in muscle tissue, including but not limited to smooth, skeletal and cardiac muscles of the human or animals. The compositions for intramuscular administration can comprise the block copolymers of poly(oxyethylene) and poly(oxypropylene).

In still another preferred embodiment, the invention relates to compositions having at least one poly(oxyethylene) and poly(oxypropylene) block copolymer with oxyethylene content of 50% or less, and at least one poly(oxyethylene) and poly(oxypropylene) block copolymer with oxyethylene content of 50% or more, combined with a polyanionic polymer and a polynucleotide. The preferable ratio by weight of the block copolymer with oxyethylene content of 50% or less to the block copolymer with oxyethylene content of 50% or more is 1:2, more preferably 1:5.

It is preferred that the compositions of this invention do not form gels. The dispersions include suspensions, emulsions, microemulsions, micelles, polymer complexes, and real polymers solutions are particularly preferred. In one aspect the concentration of the polymers and block copolymers in the polynucleotide compositions is less that 10%, preferably less that 1%, more preferred less than 0.5%, yet more preferred less than 0.1%.

Block copolymers are most simply defined as conjugates of at least two different polymer segments (Tirrel, M., Interactions of Surfactants with Polymers and Proteins, Goddard E. D. and Ananthapadmanabhan, K. P. (eds.), CRC Press, Boca Raton, Ann Arbor, London, pp. 59-122, (1992). Some block copolymer architectures are below.

Block Copolymer Architecture
(Circles indicate joints of polymer segments)

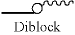

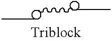

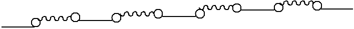

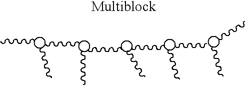

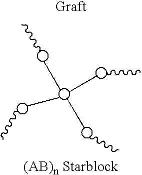

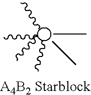

The simplest block copolymer architecture contains two segments joined at their termini to give an A-B type diblock. Consequent conjugation of more than two segments by their termini yields an A-B-A type triblock, A-B-A-B- type multiblock, or even multisegment A-B-C- architectures. If a main chain in the block copolymer can be defined in which one or several repeating units are linked to different polymer segments, then the copolymer has a graft architecture of, e.g., an A(B)ₙ type. More complex architectures include for example (AB)ₙ or AₙBₘ starblocks which have more than two polymer segments linked to a single center.

Formulas XVIII-XXIII of the invention are diblocks and triblocks. At the same time, conjugation of polycation segments to the ends of polyether of formula XVII yields starblocks (e.g., (ABC)₄ type). In addition, the polyspermine of examples 13-15 (below) are branched. Modification of such a polycation with poly(ethylene oxide) yields a mixture of grafted block copolymers and starblocks. In accordance with the present invention, all of these architectures can be useful for polynucleotide delivery.

In another aspect, the invention provides a polynucleotide complex between a polynucleotide and polyether block copolymers. Preferably, the polynucleotide complex will further include a polyanionic polymer. The compositions can further include suitable targeting molecules and surfactants. In another aspect, the invention provides a polynucleotide complex between a polynucleotide and a block copolymer comprising a polyether block and a polycation block. In yet another aspect, the invention provides polynucleotides that have been covalently modified at their 5′ or 3′ end to attach a polyether polymer segment.

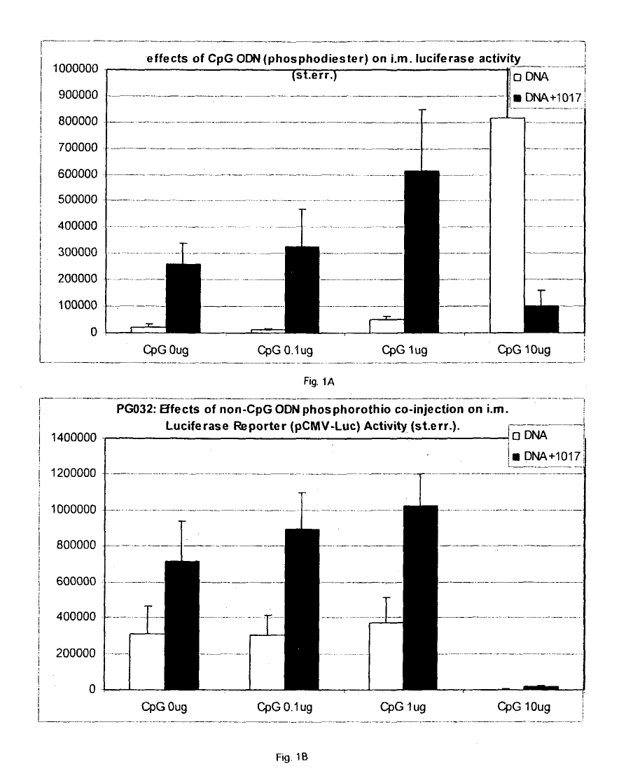

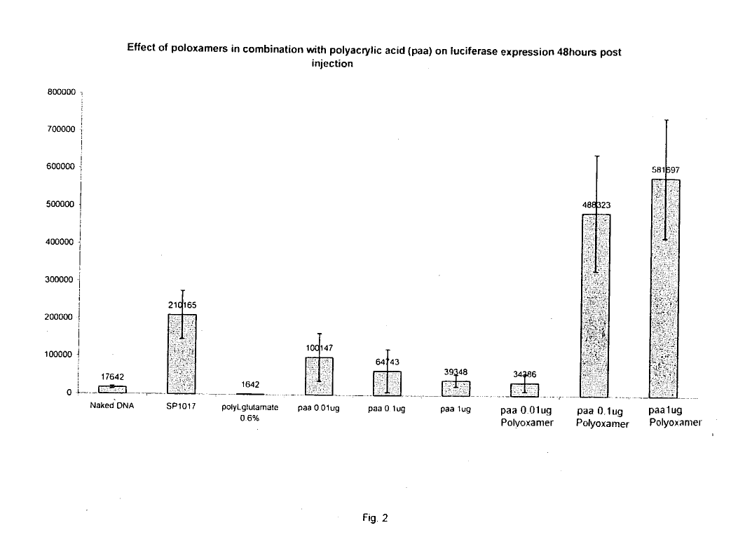

It has been found that gene expression was improved when a plasmid DNA was combined to phosphodiester and phosphorothioate oligos (CpG and non CpG containing oligos), as shown in FIG. 1A and FIG. 1B. It has been found that poloxamers enhanced further the effect of the oligos. It was further found that using a polymer that contained multiple negative charges, such as acrylic acid had an improved effect on gene therapy. Accordingly, polyacrylic acid by itself increased gene expression over naked DNA and poloxamers with polyanionic fragments had an additive or synergistic effect as shown in FIG. 2.

Further, any range of numbers recited in the specification or paragraphs hereinafter describing or claiming various aspects of the invention, such as that representing a particular set of properties, units of measure, conditions, physical states or percentages, is intended to literally incorporate expressly herein by reference or otherwise, any number falling within such range, including any subset of numbers or ranges subsumed within any range so recited. The term “about” when used as a modifier for, or in conjunction with, a variable, is intended to convey that the numbers and ranges disclosed herein are flexible and that practice of the present invention by those skilled in the art using temperatures, concentrations, amounts, contents, carbon numbers, and properties that are outside of the range or different from a single value, will achieve the desired result, namely, compositions containing polynucleotides and block copolymers as well as methods of inserting polynucleotides into cells in humans and animals.

It is to be understood that the above-described embodiments are illustrative of only a few of the many possible specific embodiments which can represent applications of the principles of the invention. Numerous and varied other arrangements can be readily devised in accordance with these principles by those skilled in the art without departing from the spirit and scope of the invention.

What is claimed is:

1. A composition comprising (a) a polynucleotide or its derivative thereof, (b) at least one polyanionic polymer, and (c) at least one amphiphilic block copolymer.

2. The composition of claim 1, wherein said at least one amphiphilic block copolymer is a block copolymer of poly(oxyethylene) and poly(oxypropylene).

3. The composition of claim 2, wherein said at least one amphiphilic block copolymer contains at least one poly(oxyethylene) and poly(oxypropylene) block copolymer with oxyethylene content of 50% or less, and at least one poly(oxyethylene) and poly(oxypropylene) block copolymer with oxyethylene content of 50% or more.

4. The composition of claim 1, wherein said amphiphilic block copolymer is selected from the group of block copolymers having the formulae (I), (II), (III) and (IV):
A-B-A′, (I)
A-B, (II)
B-A-B′ and (III)
L(R′)(R²)(R³)(R⁴) (IV)
wherein A and A′ are A-type linear polymeric segments, B and B′ are B-type linear polymeric segments, and R¹, R²,R³, and R⁴ are either block copolymers of formulas (I), (II), or (III), or hydrogen and L is a linking group, with the proviso that no more than two of R¹, R², R³, or R⁴ are hydrogen.

5. The composition of claim 1, wherein said anionic polymer has a plurality of anionic repeating units.

6. A polynucleotide composition comprising (a) a polynucleotide or derivative thereof and (b) a block copolymer having a polyether segment and a polyanion segment.

7. The composition of claim 6, further comprising a polyanionic polymer.

8. The composition of claim 6, wherein said block copolymer is selected from the group of polymers having the formulae (V-a), (VI-a), (VII), (VIII-a), (V-b), (VI-b), (VIII-b), (VIII-c) and (VIII-d):
B-A-R, (V-a)
A-R, (VI-a)
A-R-A′, (VII)
R-A-R′, (VIII-a)
A-B-R, (V-b)
A-R-B, (VI-b)
R-A-B, (VIII-b)
R-A-B-A, and (VIII-c)
R-A-B-A-R; (VIII-d)
wherein A, A′, and B are as described above, wherein R and R′ are polymeric segments having a plurality of anionic repeating units, and each anionic repeating unit in a segment is the same or different from another unit in the segment.

9. The composition of claim 6, wherein said polyanion segments are combined with poloxamers and comprises at least three of the same or different repeating unites containing at least one atom selected from the group consisting of oxygen, sulfur, or phosphorus.

10. The composition of claim 6, wherein said polyanion segment is selected from the group consisting of polymethacrylic acid and its salts, polyacrylic acid and its salts, copolymers of methacrylic acids and its salts, copolymers of acrylic acid and its salts, heparin, poly(phosphate) and polyamino acid.

11. A method of delivering a polynucleotide or its derivative to a cell comprising administering a composition of claim 1.

12. A method of delivering a polynucleotide or its derivative to a cell comprising administering a composition of claim 6.

13. A method of treating a human or an animal comprising administering the composition according to claim 1.

14. The method of claim 13, wherein the composition is administered orally, topically, rectally, vaginally, parenterally, intramuscularly, intradermally, subcutaneously, intraparitoneally, or intravenously, or by pulmonary route by use of an aerosol, or parenterally.

15. The method of claim 13, wherein said composition is administered to at least one of smooth, skeletal, and cardiac muscles.

16. A method of treating a human or an animal comprising administering the composition according to claim 6.

17. The method of claim 16, wherein the composition is administered orally, topically, rectally, vaginally, parenterally, intramuscularly, intradermally, subcutaneously, intraparitoneally, or intravenously, or by pulmonary route by use of an aerosol, or parenterally.

18. The method of claim 16, wherein said composition is administered to at least one of smooth, skeletal, and cardiac muscles.

19. A method of treating a human or an animal comprising intradermally administering the composition of claim 1.

20. A method of treating a human or an animal comprising intradermally administering the composition of claim 6.
